(19) United States
(12) Patent Application Publication
(10) Pub. No.: US 2020/0016295 A1
(43) Pub. Date: Jan. 16, 2020

(54) INJECTABLE SCAFFOLDS FOR SOFT TISSUE REPAIR

(71) Applicant: LIFEBOND, LTD., Caesarea Industrial Park (IL)

(72) Inventors: Guy TOMER, Caesarea Industrial Park (IL); Aurelie BENADDI, Caesarea Industrial Park (IL); Mary HAINRICHSON, Caesarea Industrial Park (IL); Moriah ANOUCHI, Caesarea Industrial Park (IL); Amir HADID, Caesarea Industrial Park (IL); Ofer YATZIV, Caesarea Industrial Park (IL)

(21) Appl. No.: 16/508,343

(22) Filed: Jul. 11, 2019

Related U.S. Application Data
(60) Provisional application No. 62/696,904, filed on Jul. 12, 2018.

(51) Int. Cl. A61L 27/22; A61L 27/18; A61L 27/26; A61L 27/54; A61L 27/24; A61L 27/20; A61L 27/56; A61L 27/58

(52) U.S. Cl. CPC A61L 27/222; A61L 27/18; A61L 27/26; A61L 27/22; A61L 27/54; A61L 2430/34; A61L 27/20; A61L 27/56; A61L 27/58; A61L 2400/06; A61L 27/24

(57) ABSTRACT

An injectable scaffold made from biocompatible materials. The biocompatible materials comprise a biopolymer, a porogen and a cross-linking agent. The biopolymer may optionally comprise a polypeptide or a polysaccharide.

### FIELD OF THE INVENTION

The present invention relates to an injectable scaffold for soft tissue repair, and in particular to such scaffolds that comprise an enzymatically crosslinked biopolymer matrix.

### BACKGROUND OF THE INVENTION

Three-dimensional (3-D) porous scaffold plays a crucial role in tissue engineering. It serves as a temporary template to improve cell growth and new tissue formation and provides adequate porosity for nutrients and oxygen transport. Ideally, the scaffolding materials should also be a mimic of the natural extracellular matrix (ECM) of the target tissue by integrating suitable mechanical, structural, and biological signals into scaffold. To this end, a wide range of natural biopolymer-based scaffolds have been proposed for tissue repair or regeneration during the past years, including gelatin and polysaccharides (e.g. chitosan, alginate, and hyaluronic acid).

Gelatin is a partially degraded product of collagen, which is the major protein component of natural ECM (extracellular matrix). It has been blended with other organic or inorganic biomaterials to fabricate 3-D scaffolds for various tissue engineering applications.

Polysaccharides are natural biomaterials which are inexpensive, and most of them are easily degradable. Polysaccharides are usually non-toxic, biocompatible and show a number of physico-chemical properties that make them suitable for different applications in cell therapy and tissue regeneration.

Chitosan is a polysaccharide common in the biomedical field due to its biocompatibility and low toxicity. It is a nitrogen-containing polysaccharide and related chemically to cellulose. Chitosan also promoted tissue formation and remodeling of damaged tissues in large, open wounds. It has been shown that a chitosan hydrogel interacts with fibroblast growth factor-2 in open wounds of diabetic mice, resulting in increased wound closure rate.

Alginates are linear unbranched polysaccharides. It is biodegradable and has controllable porosity. Due to their hemostatic properties, alginate and its salts are used for wound treatment in various forms such as gel or sponge. Calcium alginate can also increase cellular activity properties such as adhesion and proliferation.

Hyaluronic acid is abundant in skin, cockscomb, cartilage, and vitreous humor. This biomaterial has a high capacity of lubrication, water sorption, and water retention and influences several cellular functions such as migration, adhesion, and proliferation.

### SUMMARY OF THE INVENTION

The background art does not suggest a solution for soft tissue repair, based on biocompatible materials that are non-toxic, non-inflammatory and biodegradable, yet that can form an injectable scaffold.

The present invention overcomes these deficiencies of the background art by providing an injectable scaffold made from biocompatible materials.

The biocompatible materials comprise a biopolymer, a porogen and a cross-linking agent. The biopolymer may optionally comprise a polypeptide or a polysaccharide. The polypeptide is optionally selected from the group consisting of gelatin or gelatin related variants.

The polysaccharide may optionally comprise a homoglycan or a heteroglycan.

Non-limiting examples of homoglycans include starch, β-Glucans, chitin, chitosan, cellulose and its derivatives, pullulan, dextran. Non-limiting examples of heteroglycans include alginic acid (alginate), glycosaminoglycans (heparin, heparin sulfate, chondroitin sulfate, hyaluronic acid), pectins, gaur gum, xanthan gum.

The polysaccharide is preferably selected from the group consisting of β-glucan, cellulose, alginic acid, hyaluronic acid, chitin, chitosan, heparin, heparan sulfate, chondroitin sulfate, dermatan sulfate, keratan sulfate, glycosaminoglycans, pectin, dextran, and starch.

The porogen promotes pore formation. Precipitation or particulate leaching is one of the most straightforward methods for pore formation. Each particle able to leave the gelatin matrix can be considered as a porogen. The porogen optionally is selected from the group consisting of a polysaccharide, poly(styrene), a glycol based polymer with different MW, salts (e.g. sodium chloride), supercritical carbon dioxide, and polymeric spheres with controlled size.

Non-limiting examples of the glycol based polymer include poly(ethylene glycol) (PEG), poly(propylene glycol) (PPG).

Non-limiting examples of salts include KCl, NaCl, Na₂SO₄, NaHCO₃, and K₂HPO₄, pyridinium and imidazolium salts.

The polysaccharide is optionally selected from the group consisting of β-glucan, cellulose, alginic acid, hyaluronic acid, chitin, chitosan, heparin, heparan sulfate, chondroitin sulfate, dermatan sulfate, keratan sulfate, glycosaminoglycans, pectin, dextran, and starch.

PEG may have a molecular weight range as given in the table below. PEG is optionally selected from the group consisting of PEG1450 and PEG4000.

Preferably the biocompatible materials feature an enzymatically crosslinked gelatin matrix and a porogen, such that the biopolymer comprises gelatin and the cross-linking agent comprises transglutaminase. Cross-linking gelatin takes advantage of the adhesive properties of gelatin, by stabilizing the gelatin matrix and rendering it thermally stable in body temperature.

More preferably said transglutaminase comprises microbial transglutaminase (mTG). Optionally transglutaminase is present at a concentration of from 0.0006 to 2 mg transglutaminase/cm³ gelatin matrix.

Optionally the gelatin is present in a weight/weight percentage of 1-15%, preferably in a weight/weight percentage of 5-10% and more preferably in a weight/weight percentage of 7-9%.

Preferably the porogen comprises one or more of PEG, alginate, chitosan or hyaluronic acid.

Preferably the porogen is PEG in an amount of 1-10%, more preferably 2-7%. Optionally and preferably if the porogen is PEG, then the porogen comprises PEG4000. More preferably, the porogen comprises PEG4000 in an amount of 1-10%, more preferably 2-7%.

If the porogen comprises alginate, chitosan or hyaluronic acid, more preferably the porogen also comprises PEG.

Some non-limiting examples of ranges of materials are given below.

| | | | | More |
|---|---|---|---|---|
| | | Optional | Preferable | preferable |
| Materials | Parameter | range | range | range |
| Gelatin | bloom | 50-325 | — | 225-325 |
| | Concentration (%) | 1-20 | 5-15 | 8-10 |
| mTG/PEG- | concentration | 0.5-10 | 1-5 | 1.5-2.5 |
| mTG | (U/mL) | | | |
| chitosan | Viscosity (mPas) | 5-500 | 10-200 | 70-150 |
| | Deacetylation | 70-95 | 75-90 | 80-85 |
| | degree (%) | | | |
| | Concentration (%) | 0.1-1.5 | 0.5-1 | 0.8 |
| Alginate | Concentration (%) | 1-6 | 4-6 | 6 |
| hyaluronic | Concentration (%) | 0.1-1.5 | 0.5-1 | 0.7 |
| acid | | | | |
| PEG | Molecular weight | 400-10000 | 1000-5000 | 1450-4000 |
| | (Da) | | | |
| | Concentration (%) | 0.5-10 | 1-8 | 4-6 |

* The concentrations are the FINAL concentrations, after components mixing. The concentration in the component is two-fold higher, when the enzyme and gelatin components are mixed 1:1.

Some non-limiting examples of terms and abbreviations are provided below:
HA—hyaluronic acid
PEG—polyethylene glycol
mTG—microbial transglutaminase
PEG-mTG—modified mTG, modified by PEG molecules
HPMC—hydroxypropyl methylcellulose
NaOH—sodium hydroxide

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIG. 1 shows a non-limiting example of a collagen sample;

FIGS. 2A-2B show the morphologies of (ESEM): (a) LifeSeal; (b) 8% gelatin crosslinked by 2.5 U/mL PEG-mTG;

FIGS. 3A-3C show the morphologies of 8% gelatin containing: (a) 0.8% chitosan; (b) 0.7% hyaluronic acid; (c) 3% alginate;

FIGS. 4A-4E show the morphologies of 8% gelatin containing: (a) 5% PEG1450 in gelatin component; (b) 5% PEG1450 in enzyme component; (c) 5% PEG4000 in gelatin component; (d) 2.5% PEG4000 in enzyme component; (e) 5% PEG4000 in enzyme component;

FIGS. 5A-5C show the morphologies of 8% gelatin containing: (a) PEG4000 in enzyme component and hyaluronic acid; (b) PEG4000 in enzyme component and alginate; (c) PEG1450 in enzyme component and alginate;

FIG. 6 shows the rate of cell growth in a 2D culture;

FIG. 7 shows the digestion profile of gelatin-based hydrogels in trypsin solution;

FIG. 8 shows the digestion profile of gelatin-based hydrogels in collagenase solution;

FIG. 9 shows the digestion profile of gelatin-based hydrogels in trypsin solution; and

FIG. 10 shows a diagram representing the porosity and the adhesive strength of the studied formulations, and acceptance criteria.

### DESCRIPTION OF PREFERRED EMBODIMENTS

The present invention is of an injectable scaffold made from biocompatible materials.

To support cell ingrowth and facilitate the exchange of nutrients and cellular waste products, the in situ formed injectable scaffolds preferably possess highly porous networks with specified pore morphology. In this respect, parameters for at least some embodiments of the scaffold include pore size, porosity and interconnectivity of the porous network. To fabricate porous injectable scaffolds, several methods of in situ pore formation have been developed including leaching, gas forming and atomization.

As a non-limiting example demonstrated herein, the leaching method was adopted by introducing water-soluble PEG which could serve as polymeric porogen and was expected to promote the generation of porous structures.

Experimental data provided below compares different formulations of injectable scaffolds and investigates the influence of the composition on 3 main features: morphology, mechanical properties and swelling capacity. Preferred formulations meet the following criteria:
Nontoxic and sterile components
Injectability
Mechanical strength and resistance to in situ forces
Biodegradation
Pore morphology
Incorporation of bioactive molecules

This present invention, in at least some embodiments, demonstrates a feasible strategy to fabricate porous, adhesive, injectable crosslinked gelatin scaffolds with a high adhesive strength and an interconnected porous structure in order to promote the cell adhesion capacity for tissue engineering.

According to some embodiments of the present invention, there is provided an injectable scaffold in which the cross-linking material comprises transglutaminase and the cross-linkable protein comprises gelatin.

Suitable gelatin and transglutaminase can be obtained by any of the methods known and available to those skilled in the art. Gelatin may optionally comprise any type of gelatin which comprises protein that is known in the art, preferably including but not limited to gelatin obtained by partial hydrolysis of animal tissue and/or collagen obtained from animal tissue, including but not limited to animal skin, connective tissue (including but not limited to ligaments, cartilage and the like), antlers or horns and the like, and/or bones, and/or fish scales and/or bones or other components; and/or a recombinant gelatin produced using bacterial, yeast, animal, insect, or plant systems or any type of cell culture.

According to preferred embodiments of the present invention, gelatin from animal origins preferably comprises gelatin from mammalian origins and more preferably comprises one or more of pork skins, pork and cattle bones, or split cattle hides, or any other pig or bovine source. More preferably, such gelatin comprises porcine gelatin since it has a lower rate of anaphylaxis. Gelatin from animal origins may optionally be of type A (Acid Treated) or of type B (Alkaline Treated), though it is preferably type A.

Preferably, gelatin from animal origins comprises gelatin obtained during the first extraction, which is generally performed at lower temperatures (50-60° C., although this exact temperature range is not necessarily a limitation).

The transglutaminase may optionally comprise any plant, animal, or microbe derived transglutaminase. Preferably the transglutaminase derived from Streptoverticillium mobaraensis is used.

The transglutaminase may optionally be in a composition comprising at least one other substance, such as a stabilizer or filler for example. Non-limiting examples of such materials include maltodextrin, hydrolyzed skim milk protein or any other protein substance, sodium chloride, safflower oil, trisodium phosphate, sodium caseinate or lactose, or a combination thereof preferably other than blood derived Factor XIII.

Although the optimal pH for activity of crude transglutaminase is 6.0, it also functions with high activity in the range of pH 5.0 to pH 8.0. Therefore, a composition according to the present invention for implant fixation preferably has a pH value in a range of from about 5 to about 8.

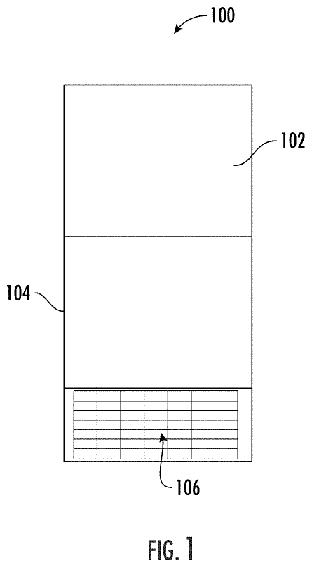

FIG. 1 shows a non-limiting example of a sample scaffold, which may be for example from collagen. As shown, a scaffold 100 features a collagen support structure portion 102, which may also comprise a second portion 104. Each portion 102 and 104 is preferably about 1-3 cm in length, and is more preferably about 2 cm in length. Each portion 102 and 104 is optionally from about 20% to 75% of the length of the total support structure, but is preferably 40-60% of the length.

Scaffold 100 also preferably comprises an adhesive section 106, which may also comprise a support section from collage with an adhesive layer, such as a cross-linked gel layer as described herein. Adhesive section 106 may comprise the cross-linked gel as described herein, optionally without a further support section from collagen. Adhesive section 106 is optionally from 0.25 to 2 cm in length, preferably 1 cm in length.

The width of scaffold 100 is optionally from 0.5 cm to 5 cm, preferably 1-3 cm and more preferably 2.5 cm.

The total length of scaffold 100 is optionally from 1 cm to 10 cm, preferably 3-7 cm and more preferably 5 cm.

### EXAMPLES

### Example 1

### 1.1 Materials

Gelatin powder (lot #604308)
Chitosan powder (LB #1106)
PEG-mTG (PE 1701)
PEG1450 (LB #826)
PEG4000 (LB #1144)
Sodium acetate (Na—Ac) buffer, pH 6, 20 mM
Sodium citrate (Na-Cit) buffer, pH 6, 1.5M
HPMC 2.5% (preparation according to WI-01-0015).
Acetic acid (glacial, LB #800)
NaOH 4M solution
Alginate powder (LB #980)
Hyaluronic acid (LB #1149)
Saline 0.9%
Trypsin (LB #936)
Collagenase (LB #841)

### 1.2 Tools

Stirrer and hot plate (LB-HS22-07)
5 mL syringes
Three way stopcocks
Analytic balance (LB-SC-06)
Instron (model 3343)
Incubator (LB-MI18-02)
Shaker incubator (LB-SI10-02)
Spectrophotometer (LB-SP4-02)
pH-meter (LB-PM12-03)

### 1.3 Solutions Preparation

16% gelatin: 38 gr gelatin were dissolved in 200 mL Na—Ac buffer during heating and stirring (using a Teflon coated magnetic stir bar). The solution was transferred to 5 mL-syringes.
16% gelatin+10% PEG1450 or PEG4000: 16% gelatin was prepared as described above. 8.4 gr of PEG were dissolved in 100 mL of 16% gelatin solution, during stirring and heating. The solution was transferred to 5 mL-syringes.
5 U/mL PEG-mTG was prepared by mixing 30 mL HPMC 2.5% with 13.6 mL water. 6 mL Na-Cit buffer were slowly added and mixed until a homogenous solution was obtained. Finally, 407 microliters were added and the solution was mixed for 2 min. After a clear solution (without air bubbles) was obtained, the solution was transferred to 5 mL-syringes.
5 U/mL PEG-mTG+1.6% chitosan: first, 0.8 gr chitosan were dissolved in 50 mL acetic acid 1% (49.5 mL water+0.5 mL acetic acid) during heating to −50° C. and stirring (using a Teflon coated magnetic stir bar). After a clear solution was obtained, the pH was adjusted to 5.5 by titration using NaOH 4M (1 mL). 0.163 mL PEG-mTG were added to 20 gr of chitosan solution (1.6%, pH 5.5). The solution was transferred to 5 mL-syringes.
5 U/mL PEG-mTG+6% alginate: first, a 15% alginate solution in 0.45M buffer citrate was prepared by mixing 10 mL Na-citrate buffer (1.5M, pH 6) with 23 mL water and dissolving 5 gr alginate during heating and continuous stirring. 4 gr of this alginate solution were mixed with 6 mL HPMC solution (2.5%) and 81 microliter PEG-mTG were added.
5 U/mL PEG-mTG+1.5% hyaluronic acid: a 15 mg/mL HA solution was prepared by dissolving 150 mg HA in a mixture of water (8.8 mL) and sodium citrate buffer (1.2 mL). Finally, 81 microliter PEG-mTG were added.
5 U/mL PEG-mTG+10% PEG1450 or 10% PEG4000: first a 37% PEG solution was prepared by dissolving 11.1 gr PEG in 30 mL water, during stirring and heating. 2.7 mL of the PEG solution were mixed with 6 mL HPMC solution (2.5%) and 1.2 mL Na-cit buffer. Finally, 81 microliter PEG-mTG were added.
5 U/mL PEG-mTG+5% PEG4000: first, a 55% PEG4000 solution was prepared by dissolving 11 gr PEG4000 in 20 mL water during stirring and heating. Then, 6 mL HPMC, 1.2 mL Na-cit buffer, 1.81 mL water and 0.91 mL PEG4000 were mixed together until getting a homogenous solution. Finally, 81 microliter PEG-mTG were added.
5 U/mL PEG-mTG+10% PEG4000+1.6% chitosan: first, a 2% chitosan solution was prepared by dissolving 0.4 gr chitosan in 1% acetic acid, during heating and stirring. The pH was adjusted to 5.5 by titration with NaOH 4M. Then, a 50% PEG4000 solution was prepared by dissolving 10 gr PEG4000 in 20 mL water, during heating and stirring. Finally, 8 mL chitosan solution were mixed with 2 mL PEG4000 solution and 81 microliter PEG-mTG were added.
5 U/mL PEG-mTG+10% PEG1450 or 10% PEG4000+6% alginate: first, a 15% alginate solution was prepared by dissolving 3 gr alginate in a mixture of water and Na-citrate buffer (1:1). A 60% PEG solution was prepared by dissolving 12 gr PEG in 20 mL water during heating and stirring. Then, 2.4 mL of the alginate solution (15%) were mixed with 6 mL of HPMC (2.5%) and 1.6 mL of the PEG solution (60%). Finally, 81 microliter PEG-mTG were added.
5 U/mL PEG-mTG+10% PEG4000+1.5% hyaluronic acid: first, a 11.5% PEG4000 solution was prepared by dissolving 4.6 g PEG4000 in 40 mL water. Then, 0.3 g HA were dissolved in a mixture of 17.6 g of the PEG4000 solution and 2.4 mL Na—Ac buffer. Finally, 0.16 mL PEG-mTg were added.

### 1.4 Gels Preparation:

The syringes (gelatin and enzyme components) were placed in a water bath at 35° C. for 2 hours. The components were mixed with a volumetric ratio of 1:1 using a metal applicator and a static mixer. Curing occurred at 37° C. for 30 min, afterward saline was added to the gels. For the formulations containing alginate, a saline solution containing 50 mM CaCl₂) was used.

The formulations that have been tested are:
Life-Seal (Batch 15033-15038)
16% gelatin crosslinked by 5 U/mL PEG-mTG
16% gelatin crosslinked by 5 U/mL PEG-mTG+1.6% chitosan
16% gelatin crosslinked by 5 U/mL PEG-mTG+1.5% hyaluronic acid
16% gelatin crosslinked by 5 U/mL PEG-mTG+6% alginate
16% gelatin+10% PEG1450 crosslinked by 5 U/mL PEG-mTG
16% gelatin+10% PEG4000 crosslinked by 5 U/mL PEG-mTG
16% gelatin crosslinked by 5 U/mL PEG-mTG+5% PEG4000
16% gelatin crosslinked by 5 U/mL PEG-mTG+10% PEG1450
16% gelatin crosslinked by 5 U/mL PEG-mTG+10% PEG4000
16% gelatin crosslinked by 5 U/mL PEG-mTG+10% PEG1450+6% alginate
16% gelatin crosslinked by 5 U/mL PEG-mTG+10% PEG4000+6% alginate
16% gelatin crosslinked by 5 U/mL PEG-mTG+10% PEG4000+1.6% chitosan
16% gelatin crosslinked by 5 U/mL PEG-mTG+10% PEG4000+1.5% hyaluronic acid

### 1.4.1 Application of the Scaffolds on Collagen

The enzyme and gelatin syringes were mixed with a volumetric ratio of 1:1 using a three way stopcock. 0.3-0.4 g of the mixture were applied on a collagen sheet (in the appropriate area) and a second collagen sheet was immediately placed above. 6 repeats of each formulation were tested. After application, the samples were placed in a petri dish, between two wet gauzes, and placed at 37° C. for 30 minutes. Finally, the gauzes were removed and 20 mL saline 0.9% were added to each dish plate (saline 0.9%+50 mM CaCl₂ was used for the samples containing alginate). The samples were kept at 37° C. overnight.

### 1.4.2 Lap Shear Test

The collagen sheets were held by the Instron's grips and extension was performed at a rate of 0.4 mm/sec (the test started after a preload of 0.2 N). From the results, the maximum load (N) and the extension

$$( \frac{Δ L}{L} * 100 = \frac{Δ L [ mm ]}{10 [ mm ]} * 100 )$$

were calculated.

### 1.5 Cell Viability in 2D Culture

The method and procedure are descripted in the report DHF-DR-0170.

### 1.6 Enzymatic Degradation

Once the gels were prepared as descripted in the section 1.4, they were washed in saline, for 24 hours at 37° C. In parallel, a trypsin or collagenase solution was prepared:
Trypsin solution

First, 5 mL of 0.1 mg/mL trypsin were prepared by dissolving 0.5 gr trypsin in 5 mL HCl 1 mM. This solution was diluted 100-fold in phosphate buffer (50 mM, pH 7.4) to obtain a final trypsin concentration of 12 U/mL.
Collagenase solution

First, a 0.5M HEPES solution, a 110 mM CaCl₂ solution and a 4 M NaCl solution were prepared. Then, 10 mL of the HEPES solution, 0.91 mL of the CaCl₂ solution and 3.75 mL of the NaCl solution were mixed and the final volume was completed to 100 mL with water (final concentrations—0.05M HEPES, 0.15M NaCl, 1 mM CaCl₂). A 15 U/mL collagenase solution was prepared by dissolving 5.2 mg collagenase in the precedent buffer solution.

Saline was replaced by the prolytic enzyme solution. The absorbance of the solution was measured at 220 nm (in order to assess the gelatin concentration) at fixed intervals. A clear enzyme solution at the same concentration was used as blank. After the total dissolution of the hydrogel, the maximal gelatin absorbance was measured and the degradation percent was calculated for each time point, as follows:

$$\% degradation ( t ) = \frac{Absorbance ( t )}{Absorbance ( max )} * 100$$

### 2 Results and Discussion

### 2.1 Morphology

### 2.1.1 Reducing the Gelatin Content

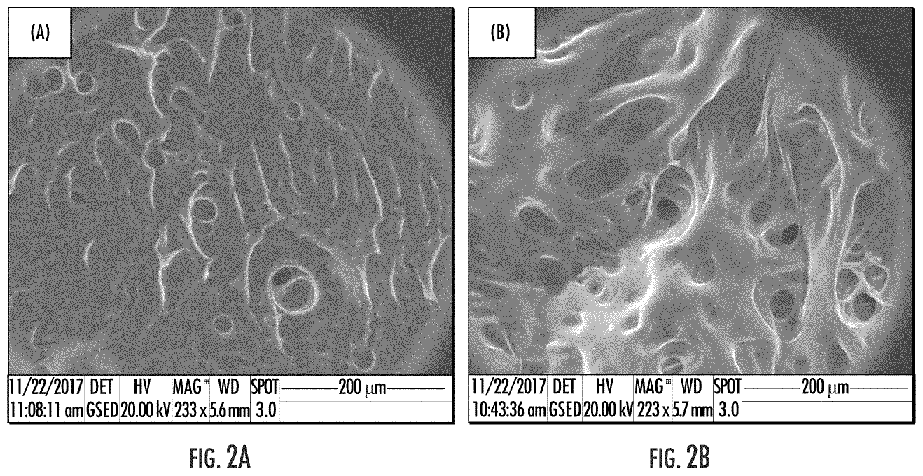

The porous structures of LifeSeal and hydrogel containing 8% gelatin (final gelatin content) are presented in FIG. 2. Both scaffolds seem to be porous with interconnected pores. The reduction of gelatin content (from 16% for LifeSeal to 8%) seems to improve the porosity and to increase the pores size (from −20 micron for Life-seal to −50 micron for the hydrogel containing 8% gelatin).

### 3 Methods

### 3.1 Morphology

The porous structures of the swollen scaffolds were visualized using secondary electron (SE) imaging in an environmental scanning electron microscope (ESEM, FEI Quanta 200). The void diameters were estimated by analyzing the low magnification SEM images. The fracture surfaces were generated using a scalpel.

### 3.2 Swelling Capacity

The scaffolds were casted in glass vials and weighted (˜1 gr) on an analytical balance. Curing occurred at 37° C. for 30 minutes, followed with addition of 15 mL saline and incubation at 37° C., with horizontal shaking at 120 rpm. After 24 hours, the hydrogels were weighted and the swelling percent was calculated as follows:

$$\% swelling = \frac{{W}_{f} - {W}_{i}}{{W}_{i}} * 100$$

Where, W_{f} and Wᵢ are the final and initial weights of the hydrogel, respectively.

### 3.3 Adhesive Strength in Lap-Shear Test

### 3.3.1 Collagen Preparation

Collagen sheets were wiped off using 70% IPA wipes, both sides of collagen were wiped well. They were placed in a beaker and covered with water, then washed in an ultrasonic bath for 3 cycles of 30 minutes each, replacing water between cycles. The washed collagen was kept in 0.9% saline until used.

The collagen sheets were cut into samples of 5 cm×2.5 cm. A mark of 2 cm was done to indicate where the Instron's top grip will be placed. A mark of 1 cm was done to indicate the area of scaffold application.

3.3.2 Addition of Polysaccharides (Chitosan. Alginate and Hyaluronic Acid)

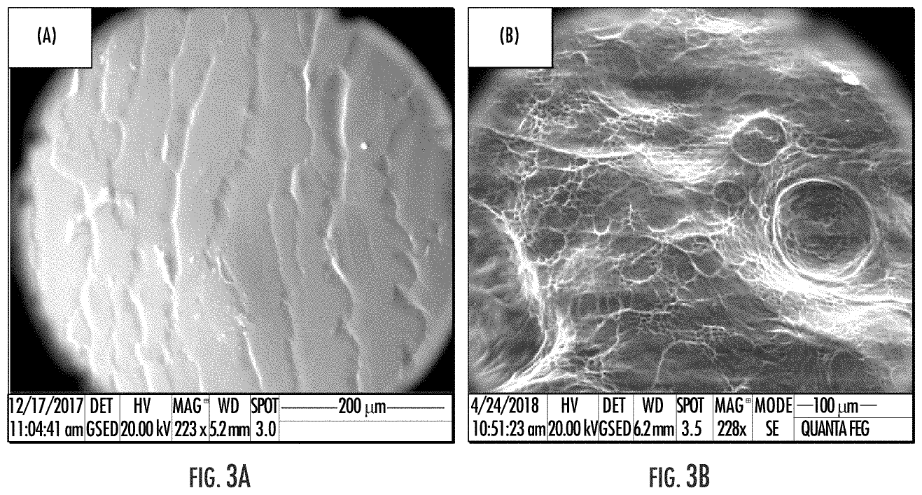

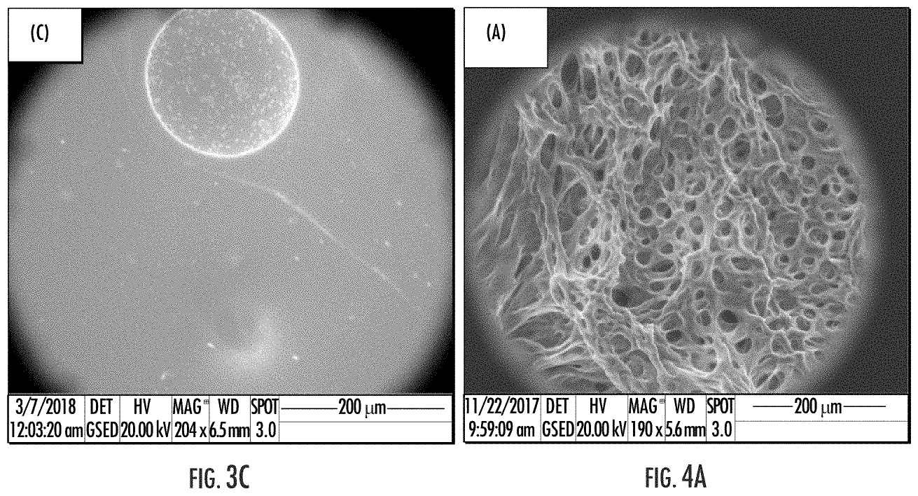

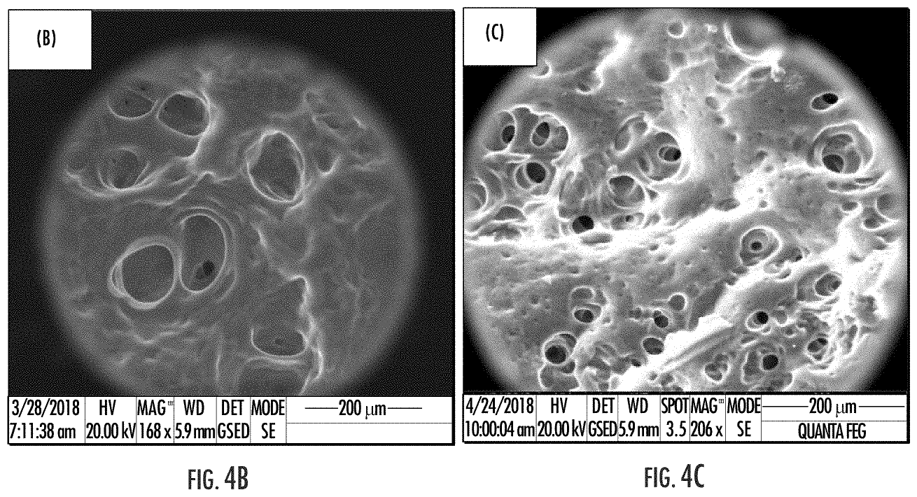

The morphologies of 8% crosslinked gelatin-based hydrogels containing chitosan, hyaluronic acid and alginate are shown in FIG. 3. The hydrogels seem to be relatively non-porous, except for hyaluronic acid which contains closed pores.

### 3.3.3 Addition of PEG as Porogen

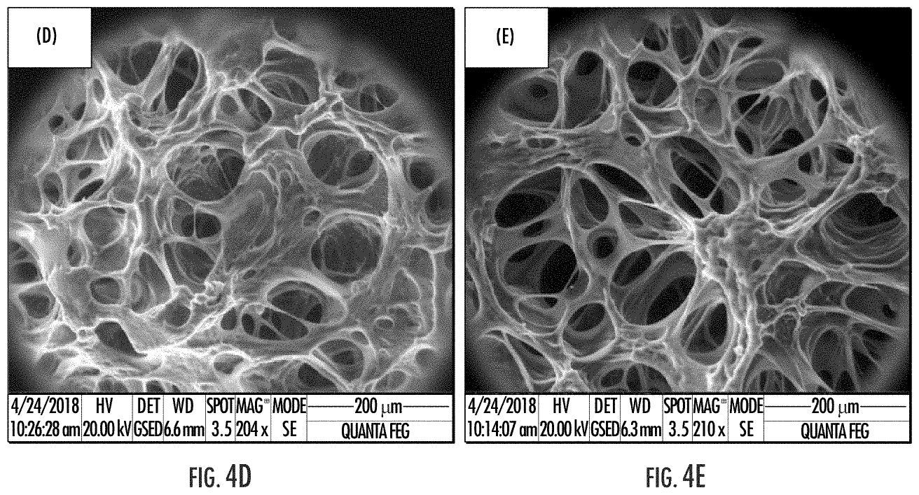

The morphologies of 8% crosslinked gelatin-based hydrogels containing PEG1450 or PEG4000 from enzyme and gelatin components are presented in FIG. 4. All the hydrogels seem to be very porous materials with interconnected pores, especially the hydrogels containing PEG1450 in gelatin component and PEG4000 in enzyme component. The main difference between these two materials is the pores size—using PEG4000 as porogen seems to lead to bigger pores comparing to PEG1450 (20-100 microns, for PEG4000 compared to 20-40 microns, for PEG1450).

### 3.3.4 Combination of PEG and Polysaccharides

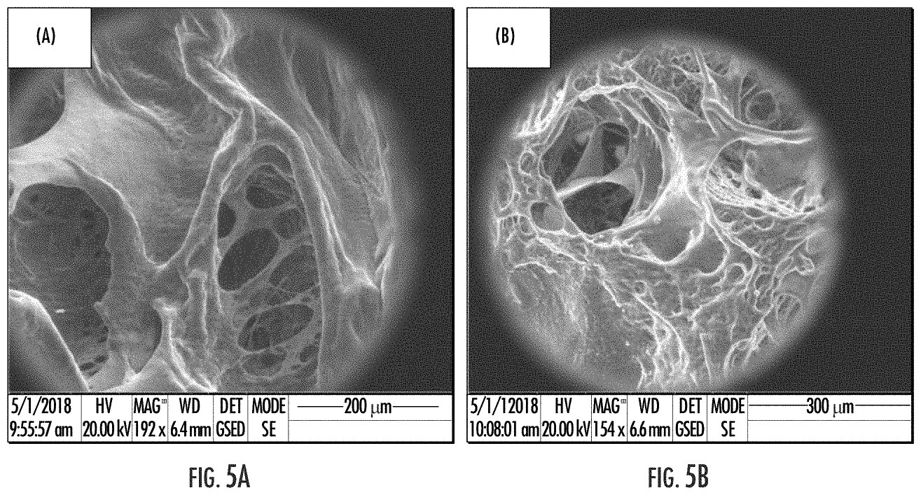

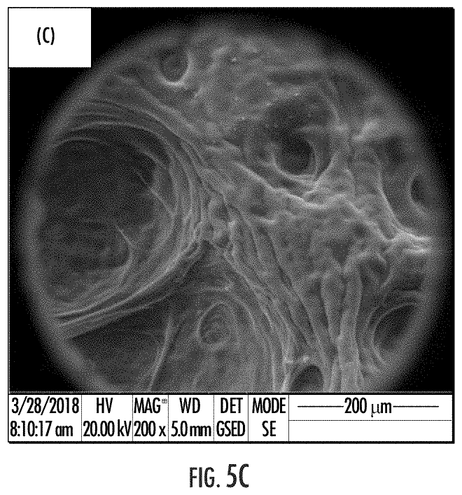

The structures of 8% based-gelatin containing PEG as porogen (in enzyme component) and hyaluronic acid or alginate are shown in FIG. 5. These materials seem to be non-homogenous. The hydrogels containing PEG4000 possess non-uniformly distributed highly porous zones with interconnected pores. The hydrogels containing PEG1450 seem to be non-porous with some closed pores.

### 3.4 Swelling Capacity

The swelling results of crosslinked-gelatin based scaffolds as a function of formulation are presented in Table 1. Overall, the swelling capacity of such hydrogels ranges from −5% to 69%. The parameters that influence the swelling capacity are: the gelatin content, presence and type of polysaccharides and porogen. The swelling capacity increases as the gelatin content decreases (the crosslink density and the chains mobility are improved, consequently the swelling capacity increases). The swelling capacity increases with the porogen (PEG) concentration. Chitosan decreases the swelling of the scaffolds. It may be related to the non-porous morphology of such hydrogels. Hyaluronic acid improves significantly the swelling capacity, probably due to the ability of HA to attract water molecules. Alginate does not seem to significantly affect the swelling capacity of such hydrogels.

TABLE 1
Swelling capacity of crosslinked-gelatin based
scaffolds as a function of formulation

| Formulation | Swelling (%) |
|---|---|
| Life-Seal (16% gelatin) | 5% |
| 8% gelatin | 10% |
| 8% gelatin + 0.8% chitosan | −5% |
| 8% gelatin + 0.7% HA | 23% |
| 8% gelatin + 3% alginate | 12% |
| 5% PEG1450 (G.C.) | 20% |
| 5% PEG1450 (E.C.) | 25% |
| 5% PEG4000 (E.C.) | 44% |
| 2.5% PEG4000 (E.C.) | 32% |
| 5% PEG1450 (E.C.) + alginate | 12% |
| 5% PEG4000 (E.C.) + alginate | 42% |
| 5% PEG4000 (E.C.) + hyaluronic acid | 69% |
| 5% PEG4000 (E.C.) + chitosan | 4% |

### 3.5 Mechanical Properties in Lap-Shear Test

The results of the mechanical properties assessed by lap-shear tests are summarized in Table 2. The maximum load ranges from 0.16 N to more than 9 N, as a function of the formulation. Some parameters seem to influence the adhesive strength of the hydrogels: chitosan seems to strengthen the matrix by up to 45%. This may be attributed to the abundancy of amino-sugar moieties in chitosan molecules which can participate in the crosslinking reaction of gelatin, increasing the crosslinking density and consequently the adhesive strength. In addition, the positively charged chitosan molecules may form electrostatic interactions with negatively charged gelatin moieties and consequently contribute to the stabilization of hydrogel and its strength. Hyaluronic acid does not seem to affect the mechanical properties, but crosslinked alginate increases the maximum load by more than three fold.

Finally, the presence of porogen (PEG1450 or PEG4000) significantly weakens the gelatin matrix (by up to 3 fold, when it is placed in the enzyme component). The combination of porogen and crosslinked alginate allows to reach the maximum load value of the basic formulation (8% gelatin).

TABLE 2
Maximum load and extension of crosslinked-
gelatin based scaffolds

| | | Extension at |
|---|---|---|
| | Maximum load | maximum load |
| Formulation | (N) | (%) |
| Life-Seal | 2.56 | 22 |
| 8% gelatin | 2.87 | 88 |
| 8% gelatin + 0.8% chitosan | 4.17 | 28 |
| 8% gelatin + 0.7% HA | 2.57 | 23 |
| 8% gelatin + 3% alginate | 9.90 | 56 |
| 5% PEG1450 (G.C.) | 0.16 | 2 |
| 5% PEG1450 (E.C.) | 0.90 | 12 |
| 2.5% PEG4000 (E.C.) | 1.10 | 11 |
| 5% PEG1450 (E.C.) + alginate | 2.56 | 24 |
| 5% PEG4000 (E.C.) + alginate | 3.47 | 30 |
| 5% PEG4000 (E.C.) + HA | 1.11 | 13 |
| 5% PEG4000 (E.C.) + chitosan | 1.54 | 16 |

### 3.6 Cell Viability in 2D Culture

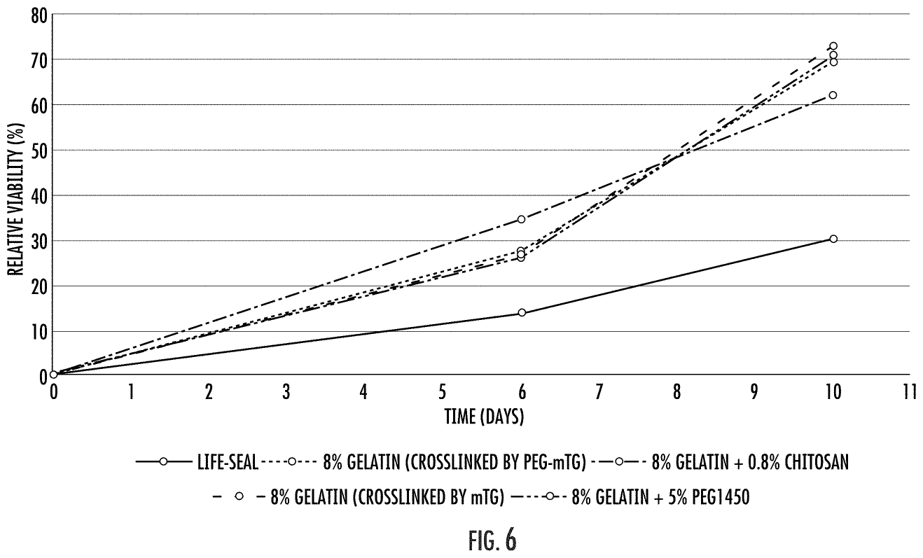

Cell viability results are shown in FIG. 6. The results demonstrate a well proliferation ability of cells from type fibroblast 3T3, on the enzymatically crosslinked gelatin-based hydrogels during 10 days of 2D culture. All the formulations seem to be suitable matrixes for 2D cell culture, except for LifeSeal that exhibits lower rate of cell growth and proliferation. It may be attributed to the higher gelatin concentration—16% vs. 8% in other formulations, or to other components present only in this item (e.g. tween 20, urea, CaCl₂).

### 3.7 Enzymatic Degradation

3.7.1 Influence of Type of Enzyme (mTG Vs. PEG-mTG) and Gelatin Concentration

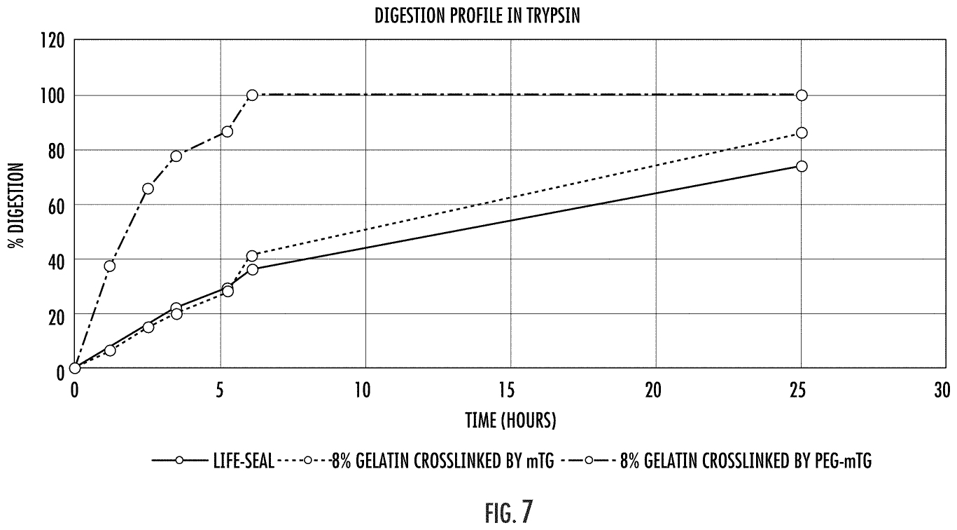

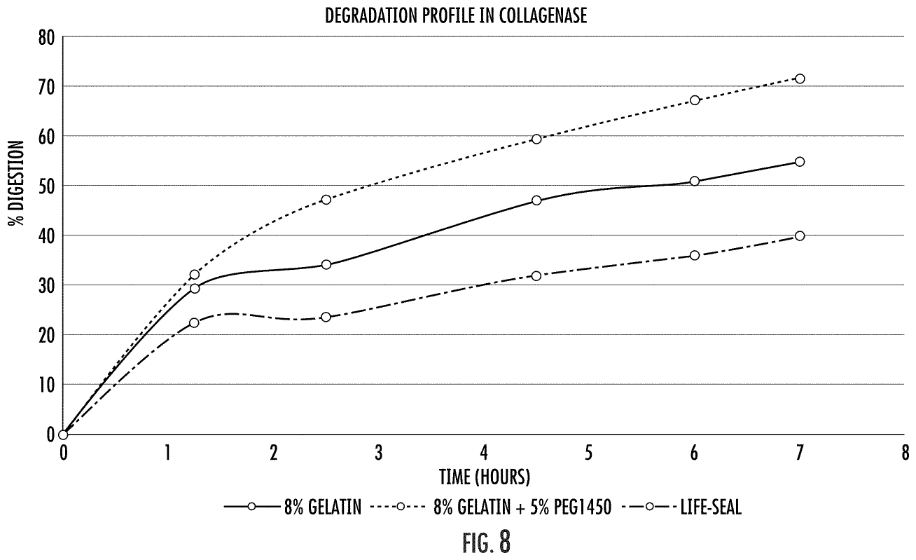

The type of enzyme and the gelatin concentration may affect the degradation profile of such scaffolds since they influence the crosslinking density. The results of this study are presented in FIG. 7. Life-Seal and 8% gelatin-based hydrogel crosslinked by mTG seem to have a similar digestion profile and their rate of degradation is significantly lower compared to 8% gelatin-based hydrogel crosslinked by PEG-mTG. As the gelatin content increases (8% vs. 16% for LifeSeal), the crosslinking density increases and consequently the degradation rate is lower. The effect of the enzyme type may be explained by steric interference of the PEG molecules in the PEG-mTG enzyme—as the steric interference is higher, the crosslinking density of the resulting hydrogel is lower and consequently the enzymatic degradation rate is higher.

### 3.7.2 Influence of Porogen and Gelatin Concentration

The effects of porogen and gelatin concentration on the enzymatic degradation profile of gelatin-based hydrogels in collagenase solution are shown in FIG. 8. LifeSeal exhibits the lower degradation rate since it contains the higher gelatin concentration (16%). The hydrogel containing PEG1450 as porogen exhibits the higher degradation rate. This trend is due to the higher porosity of such hydrogels, leading to a higher surface area and consequently the enzymatic degradation occurs faster. This results is consistent with the higher swelling capacity obtained by such hydrogels.

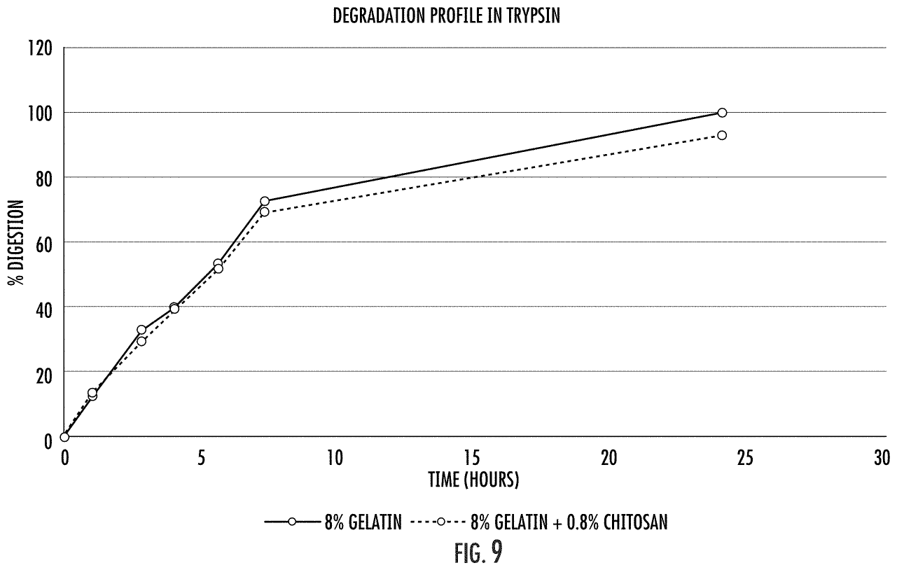

3.7.3 Influence of Chitosan The effects of chitosan on the enzymatic degradation profile of gelatin-based hydrogels in trypsin solution are shown in FIG. 9. Despite the increase in adhesive strength caused by the addition of chitosan in the 8% gelatin-based hydrogel, the latter does not seem to affect the enzymatic degradation profile of the hydrogel in trypsin solution.

### SUMMARY

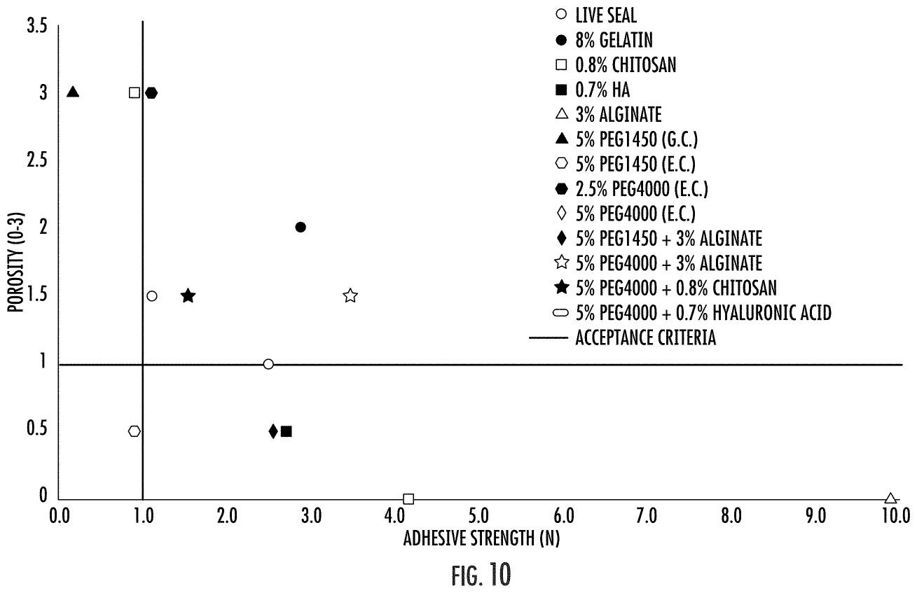

The main aim was to find gelatin-based formulations that meet the criteria to be potential injectable adhesive scaffolds for soft tissue repair (high adhesive strength, appropriate morphology, incorporation of bioactive molecules, enzymatically degradable). The results of the current study are summarized in Table 3 and in FIG. 10. It is highly challenging to develop a formulation that exhibits both high porosity and high mechanical properties. These two parameters are generally opposite—higher porosity leads to a weaker material. FIG. 10 represents a diagram of porosity vs. adhesive strength, and two acceptance criteria: minimum grade porosity of 1 (the porosity is graded from 0—non-porous material, to 3—very porous material) and minimum adhesive strength in lap-shear test of 1 N. From 13 tested formulations, only 5 meet these criteria, including 3 which contain a polysaccharide.

TABLE 3
Results summary - adhesive strength,
porosity and swelling capacity

| | Adhesive | Poros- | Swell- | Polysac- |
|---|---|---|---|---|
| | strength | ity | ing | charide |
| Formulation | (N) | (0-3) | (%) | (Yes/No) |
| Life-Seal | 2.5 | 1 | 5 | X |
| 8% gelatin | 2.9 | 2 | 10 | X |
| 0.8% chitosan | 4.2 | 0 | −5 | V |
| 0.7% HA | 2.7 | 0.5 | 23 | V |
| 3% alginate | 9.9 | 0 | 12 | X |
| 5% peg1450 (G.C.) | 0.2 | 3 | 20 | X |
| 5% peg1450 (E.C.) | 0.9 | 0.5 | 25 | X |
| 2.5% PEG4000 (E.C.) | 1.1 | 3 | 32 | X |
| 5% PEG4000 (E.C.) | 0.9 | 3 | 44 | X |
| 5% peg1450 + 3% alginate | 2.6 | 0.5 | 12 | V |
| 5% PEG4000 + 3% alginate | 3.5 | 1.5 | 42 | V |
| 5% PEG4000 + 0.8% chitosan | 1.5 | 1.5 | 4 | V |
| 5% PEG4000 + 0.7% hyal- | 1.1 | 1.5 | 69 | V |
| uronic acid | | | | |

Injectable, adhesive, porous and degradable gelatin scaffolds were successfully developed. The degradation rate by proteolytic enzyme is affected by crosslink density and matrix porosity. The incorporation of PEG molecules with different molecular weights allows to control the porous morphology and the pore size of the gelatin matrices. The addition of alginate and chitosan has a double effect—they reinforce the gelatin scaffolds and, as polysaccharides, may play an important role in would healing promotion of cell viability and proliferation. The 2D culture results demonstrate a well proliferation ability of cells on the enzymatically crosslinked gelatin-based scaffolds.

These materials are suitable injectable scaffolds for soft tissue repair according to at least some embodiments of the present invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An injectable scaffold comprising biocompatible materials, wherein the biocompatible materials comprise a biopolymer, a porogen and a cross-linking agent; wherein the biopolymer comprises a polypeptide or a polysaccharide; wherein the porogen is selected from the group consisting of a polysaccharide, poly(styrene), a glycol based polymer with different MW, salts, supercritical carbon dioxide, and polymeric spheres with controlled size.

2. The scaffold of claim 2, wherein the polypeptide is selected from the group consisting of gelatin and gelatin related variants.

3. The scaffold of claim 1, wherein the polysaccharide comprises a homoglycan or a heteroglycan.

4. The scaffold of claim 3, wherein the homoglycan comprises one or more of starch, 3-Glucans, chitin, chitosan, cellulose and its derivatives, pullulan, dextran.

5. The scaffold of claim 4, wherein the heteroglycan comprises one or more of alginic acid (alginate), glycosaminoglycans (heparin, heparin sulfate, chondroitin sulfate, hyaluronic acid), pectins, gaur gum, xanthan gum.

6. The scaffold of claim 1, wherein the polysaccharide is selected from the group consisting of 3-glucan, cellulose, alginic acid, hyaluronic acid, chitin, chitosan, heparin, heparan sulfate, chondroitin sulfate, dermatan sulfate, keratan sulfate, glycosaminoglycans, pectin, dextran, and starch.

7. The scaffold of claim 1, wherein the glycol based polymer comprises one or more of poly(ethylene glycol) (PEG), poly(propylene glycol) (PPG).

8. The scaffold of claim 1, wherein the salt comprises one or more of KCl, NaCl, Na2SO4, NaHCO3, and K2HPO4, pyridinium and imidazolium salts.

9. The scaffold of claim 1, wherein the porogen comprises a polysaccharide and the polysaccharide is selected from the group consisting of 3-glucan, cellulose, alginic acid, hyaluronic acid, chitin, chitosan, heparin, heparan sulfate, chondroitin sulfate, dermatan sulfate, keratan sulfate, glycosaminoglycans, pectin, dextran, and starch.

10. The scaffold of claim 1, wherein the porogen comprises PEG and the PEG is selected from the group consisting of PEG1450 and PEG4000.

11. The scaffold of claim 1, wherein the biocompatible materials comprise an enzymatically crosslinked gelatin matrix and a porogen, such that the biopolymer comprises gelatin and the cross-linking agent comprises transglutaminase.

12. The scaffold of claim 11, wherein said transglutaminase comprises microbial transglutaminase (mTG).

13. The scaffold of claim 12, wherein said transglutaminase is present at a concentration of from 0.0006 to 2 mg transglutaminase/cm³ gelatin matrix.

14. The scaffold of any of claims 13-15, wherein the gelatin is present in a weight/weight percentage of 1-15%, preferably in a weight/weight percentage of 5-10% and more preferably in a weight/weight percentage of 7-9%.

15. The scaffold of claim 1, wherein the porogen comprises one or more of PEG, alginate, chitosan or hyaluronic acid.

16. The scaffold of claim 15, wherein if the porogen comprises alginate, chitosan or hyaluronic acid, the porogen also comprises PEG.

17. The scaffold of claim 1, comprising a collagen support structure and an adhesive structure, connected to or integrally formed with said collagen support structure.

18. The scaffold of claim 17, wherein said adhesive structure comprises cross-linked gelatin.

19. The scaffold of claim 18, wherein said cross-linked gelatin is cross-linked with transglutaminase.

20. The scaffold of claim 18, wherein said adhesive structure forms an adhesive layer over said collagen support structure.
